(19) United States
(12) Patent Application Publication
(10) Pub. No.: US 2023/0215527 A1
(43) Pub. Date: Jul. 6, 2023

(54) TAGGING PATIENT REFERRALS

(71) Applicant: Matrixcare, Inc., Bloomington, MN (US)

(72) Inventors: Joseph CAUDILL, Bloomington, MN (US); Danny WONG, Bloomington, MN (US)

(21) Appl. No.: 18/086,375

(22) Filed: Dec. 21, 2022

Related U.S. Application Data
(60) Provisional application No. 63/295,777, filed on Dec. 31, 2021.

(51) Int. Cl. G16H 10/60; G16H 40/20; G16H 50/20; G06F 3/04855

(52) U.S. Cl. CPC G16H 10/60; G16H 40/20; G16H 50/20; G06F 3/04855

(57) ABSTRACT

Embodiments herein describe a patient evaluation system that generates tags for a patient that, e.g., an evaluator can use to determine whether a particular healthcare facility can meet the needs of the patient. The patient evaluation system can scan a patient's medical records to identify keywords or phrases that can be used to generate tags. The patient evaluation system can then assign a visual indicator to the tags to represent the tags' relevance to a particular health care facility. For example, if a tag indicates the patient needs a complex medication which the facility does not have, this tag is assigned a visual indicator a low compatibility with the healthcare facility. These tags, along with their visual indicators, can then be displayed on a graphical user interface (GUI) for the evaluator to then make a recommendation whether to transfer the patient to the healthcare facility.

### BACKGROUND

### Field

Embodiments of the present invention generally relate to generating tags for a patient and assigning a visual indicator to those tags to indicate a relationship between the tags and a healthcare facility.

### Description of the Related Art

Patients often move between different healthcare facilities such as assisted living facilities, hospitals, surgery centers, skilled nursing centers, etc. An evaluator typically has to scan the patient's medical records, which can include many pages of documents, to determine whether a particular healthcare facility is suitable for the patient. For example, the evaluator may have to read the medical records to identify the patient's medical diagnosis, the medications the patient is prescribed, a responsible payer (e.g., private insurance or government payer), and special medical equipment needed by the patient and then determine whether the healthcare facility has the equipment, staff, and resources to satisfy the patient's needs. It can take the evaluator a significant amount of time to parse the patient's records and determine whether a healthcare facility is suitable (or to select a suitable facility from a plurality of healthcare facilities operated by a healthcare provider).

### SUMMARY

Certain embodiments provide a method that includes receiving a medical record for a person, scanning the medical record to generate tags where the tags provide information about at least one of medical needs or a responsible payer of the person, assigning visual indicators to the tags to represent a compatibility of the information in the tags with a particular healthcare facility, and transmitting, for display, a GUI comprising (i) the tags along with their visual indicators and (ii) at least a portion of the medical record.

Other embodiments provide a computing system that includes a processor and memory storing an application which, when executed by the processor, performs an operation. The operation includes receiving a medical record for a person, scanning the medical record to generate tags, the tags providing information about at least one of medical needs or a responsible payer of the person, assigning visual indicators to the tags to represent a compatibility of the information in the tags with a particular healthcare facility, and transmitting, for display, a GUI comprising (i) the tags along with their visual indicators and (ii) at least a portion of the medical record.

Other embodiments provide a non-transitory computer readable medium comprising instructions to be executed in a processor, the instructions when executed in the processor perform an operation. The operation includes receiving a medical record for a person, scanning the medical record to generate tags, the tags providing information about at least one of medical needs or a responsible payer of the person, assigning visual indicators to the tags to represent a compatibility of the information in the tags with a particular healthcare facility, and transmitting, for display, a GUI comprising (i) the tags along with their visual indicators and (ii) at least a portion of the medical record.

The following description and the related drawings set forth in detail certain illustrative features of one or more embodiments.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates a block diagram of a patient evaluation system for providing tags for a patient, according to one embodiment.

FIG. 2 is a flowchart for tagging a patient's medical records, according to one embodiment.

FIG. 3 is a graphical user interface displaying tags corresponding to a patient, according to one embodiment.

FIG. 4 is a graphical user interface displaying tags corresponding to a patient, according to one embodiment.

FIG. 5 is a flowchart for assigning visual indicators to tags corresponding to a patient using a machine learning model, according to one embodiment.

FIG. 6 is a graphical user interface displaying text in a medical record used to generate a tag, according to one embodiment.

FIG. 7 illustrates a learning system for assigning visual indicators to tags, according to one embodiment.

FIG. 8 is a flowchart for assigning visual indicators to tags corresponding to a patient using a machine learning model, according to one embodiment.

FIG. 9 illustrates a block diagram of a patient evaluation system for providing tags for a patient, according to one embodiment.

FIG. 10 illustrates a computing system, according to one embodiment.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments herein describe a patient evaluation system that generates tags for a patient that an evaluator can use to determine whether a particular healthcare facility can meet the needs of the patient. The patient evaluation system can scan a patient's medical records (e.g., a Continuity of Care Document (CCD), Electronic Health Record (EHR), patient notes, etc.) to identify keywords or phrases that can be used to generate tags. For example, the system can use a keyword search or natural language processing to identify a medical diagnosis (or diagnoses) of the patient, the medications currently prescribed to the patient, responsible payer (e.g., private insurance or government payer), or special medical equipment or treatments needed by the patient (e.g., lifts or frequent computed tomography (CT) scans) to generate the tags. The patient evaluation system can then assign a visual indicator to the tags to represent the tags' relevance to a particular health care facility. For example, if a tag indicates the patient needs a complex medication which the facility does not have the expertise to administrator, this tag is assigned a visual indicator indicating the tag has a low compatibility with the healthcare facility. Coloring the tag green could indicate a high compatibility between the tag and the facility while red indicates a low compatibility with the facility. In this manner, the patient evaluation system can process the medical records to find tags that are important or relevant to the evaluator. Further, the system can add visual indicators to those tags to indicate the level of compatibility between the tag and the healthcare facility currently being considered. These tags can then be displayed on a graphical user interface (GUI) for the evaluator. The tags, along with their visual indicator, improve computer technology (i.e., the user interface) by providing a high-level synopsis of the patient's medical record and a level of compatibility with a healthcare facility, relative to a GUI that simply displays the patient's medical records without performing any textual analysis and compatibility analysis on the records.

In one embodiment, the patient evaluation system coverts the tags into selectable tags that link to portions of the medical record used to generate the tags. For example, when the patient evaluation system parses the medical record and identifies a phrase, sentence, or paragraph that causes it to generate a tag (e.g., a sentence describing the medical diagnosis of the patient), the system can tie this portion of the medical record to the tag. When displaying the GUI, the tags can be selectable tags that link to the portion of the medical record that generated the tag. When clicked or touched by the evaluator, the GUI is updated so that portion of the medical record is displayed. For example, the GUI may automatically scroll to the portion of the medical records, or a pop-up window can appear containing the portion of the medical records. The evaluator can read this portion of the medical record to learn more information about the tag and gain additional context.

In one embodiment, the patient evaluation system can use a machine learning (ML) model to assign the visual indicator to the tags. For example, the patient evaluation system can maintain a history of which patients the evaluator selected for a facility and which it did not. That is, after the patient evaluation system provides a GUI to the evaluator which includes the tags discussed above, the system can track whether the evaluator did or did not recommend the patient be moved to the healthcare facility. The ML model can then be trained using the feedback from the evaluator to learn which tags likely indicate whether the patient is or is not compatible with a healthcare facility. Once trained, when receiving a new patient referral, the patient evaluation system can input the identified tags into the ML model which can then assign the visual indicators to these tags. In this manner, the ML model can learn from past decisions made by the evaluator which tags are the most relevant to determining whether the patient should be accepted by the facility and then assign a visual indicator accordingly.

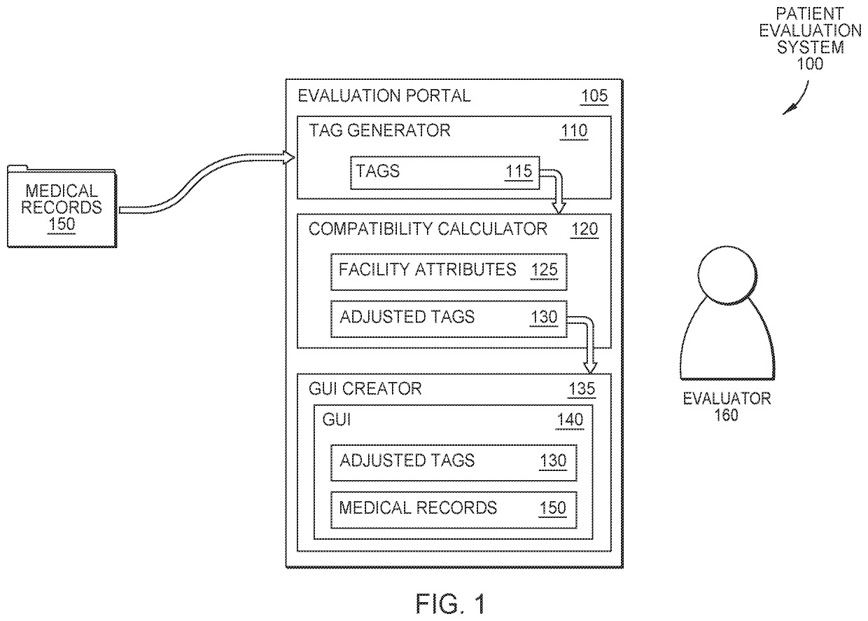

FIG. 1 illustrates a block diagram of a patient evaluation system 100 for providing tags for a patient, according to one embodiment. The patient evaluation system 100 includes a tag generator 110, a compatibility calculator 120, and a GUI creator 135, which can be software applications or modules. The tag generator 110 parses a patient's medical records 150 to generate tags 115 for the records 150. In one embodiment, an evaluator 160 transmits the medical records 150 (e.g., a CCD or EHR) to the evaluation portal 105 so that the portal 105 can provide a recommendation to the evaluator 160. The recommendation can indicate whether the patient is compatible with a particular healthcare facility, or the portal 105 can provide a recommendation indicating which facility from a pool of candidate healthcare facilities is the best fit for the patient. For example, the evaluator 160 may be tasked with identifying the healthcare facility that is most compatible with the needs of the patient. Because the patient may be referred to the evaluation portal 105 by the evaluator 160, in the discussion below the patient can also be referred to as a referral.

In another embodiment, the medical records 150 may be provided to the evaluation portal 105 by the patient or a legal guardian. For example, the patient may be looking for an assisted living center, rehabilitation center, or skilled nursing center. The patient can rely on the portal 105 to evaluate a pool of candidate healthcare facilities and provide a recommendation indicating which would be the best fit for the patient in response to factors such as the patient's medical diagnosis, prescriptions, budget, insurance, special medical equipment needed by the patient, and the like.

As discussed in more detail below, the tag generator 110 can scan the medical records to generate tags 115 for the patient. For example, the tag generator 110 may perform a keyword search or use natural language processing to identify tags indicating medical diagnoses, prescriptions, insurance, special medical equipment needed by the patient, and the like. In one embodiment, the tag generator 110 may store a list of predefined tags and then search the medical records 150 searching for a phrase or word that matches a tag in the list (or is a derivative thereof). If the medical records 150 are not in readable or editable form, the tag generator 110 may first perform an Optical Character Recognition (OCR) to, e.g., convert a PDF file or handwritten notes into a readable form.

Once the tags 115 are generated, the compatibility calculator 120 adds visual indicators to the tags 115 to generate adjusted tags 130. In one embodiment, the compatibility calculator 120 includes a list of facility attributes 125 about a facility or a pool of healthcare facilities (e.g., all the facilities in a healthcare network or all the facilities that are part of a healthcare provider). The attributes 125 can indicate which ailments or diseases a healthcare facility can treat, which prescriptions the facility can administer, the medical equipment in the facility, the lab capabilities of the facility, the expertise of the staff at the facility, the insurance accepted at the facility, and the like.

In one embodiment, the compatibility calculator 120 compares the tags 115 to the attributes 125 of one or more facilities to determine the compatibility of the patient with the facility. For example, if the patient has a tag 115 for a particular illness, the compatibility calculator 120 can determine whether the facility attributes 125 indicate the facility can treat that illness (or that type of illnesses). In another example, if the patient has a tag 115 indicating they need a special lift, the compatibility calculator 120 can determine from the attributes 125 whether that facility has the lift. In yet another example, if the patient has a tag 115 indicating her insurance, the compatibility calculator 120 can determine from the attributes 125 whether that facility accepts that insurance (e.g., whether the facility is in-network or out-of-network).

While for some tags 115 the compatibly analysis may be a simple binary result—e.g., yes, the facility does have a special lift needed by the patient, or no, the facility does not have the lift—for other tags the compatibly analysis may result in a range of values. For example, the facility may accept the patient's insurance, but it may not prefer that insurance relative to other insurers. Or the facility may have the expertise for treating a particular illness, but the facility may be primary set up to handle a different type (or types) of disease. In those situations, the compatibility between one of the tags 115 and the facility attributes 125 may be a sliding scale ranging between completely compatible (e.g., preferred) or incompatible (cannot service the patient's need).

After determining the compatibility between the tags 115 of a patient and the facility using the facility attributes 120, the compatibility calculator 120 can assign a visual indicator to the tags to represent their individual compatibility scores with the facility. For example, if the tag 115 indicates the patient needs a lift, and the facility has the lift, the tag 115 may be assigned the color green. However, if the facility did not have the lift, the tag 115 may be assigned the color red. For tags 115 where their compatibility scores are somewhere between completely compatible and incompatible, the tags 115 may be assigned the color yellow or orange. For example, if a tag 115 indicates the patient needs frequent CT scans but the facility offers CT scans on a limited time schedule or has a limited number of CT machines, this tag 115 may be assigned the color yellow. Or if the tag 115 indicates the patient has an insurance provider that is accepted, but not preferred by the facility, the tag 115 may be assigned the color yellow.

However, assigning colors to the tags 115 to generate the adjusted tags 130 is just one example of applying visual indicators. In other embodiments, the adjusted tags 130 may be assigned different luminance values or different sizes in response to their compatibility scores. In another embodiment, different highlighting or visual effects (e.g., blinking) can be applied to the adjusted tags 130 to represent their compatibility with a particular facility.

The adjusted tags 130 are then transmitted to the GUI creator 135 that is tasked to generate a GUI 140 that displays the adjusted tags 130 as well as the patient's medical records 150. The evaluator 160 (or the patient) can then use the portal 105 to view the GUI 140 that includes the adjusted tags 130. The tags 130 can provide a synopsis of the important (or most relevant) information in the medical records 150 such as the patient's medical diagnosis, prescriptions, special medical needs, etc. Further, the tags 130 have visual indicators to represent their compatibility with a particular facility.

The evaluator 160 can then make a decision on whether the patient is a good fit for a facility using solely the tags 130, or by using the tags 130 and the medical records 150. For example, if the portal 105 identified ten tags 130 and all of these tags were assigned visual indicators indicating the tags 130 are highly compatible with the facility, the evaluator 160 may recommend the patient be admitted into that facility without further evaluation of the medical records 150 in the GUI. On the other hand, if the ten tags 130 had visual indicators indicating there were all incompatible with the facility, the evaluator 160 may recommend the patient not be admitted into that facility without further evaluation of the medical records 150. If the tags 130 indicate it is a close call—e.g., five of the tags 130 indicate the facility would be a good fit while the other five tags 130 indicate the facility would not be a good fit—the evaluator 160 may spend more time evaluating the medical records 150 before making a recommendation or decision.

In one embodiment, the evaluation portal 105 includes an authentication system for ensuring only authorized individuals can access the GUI 140 which includes the patient's medical records 150. For example, the portal 105 may be accessible using a web browser, but the evaluator 160 must provide security credentials before the GUI 140 is displayed. In another embodiment, the evaluation portal 105 may be a secure application running locally (or natively) on the evaluator's computer device (e.g., a tablet, laptop, or desktop). The evaluation portal 105 may still require security credentials or biometric information (e.g., a face scan) before displaying the GUI 140 to ensure it is the evaluator 160 rather than a nefarious user who is attempting to access the patient's medical records 150.

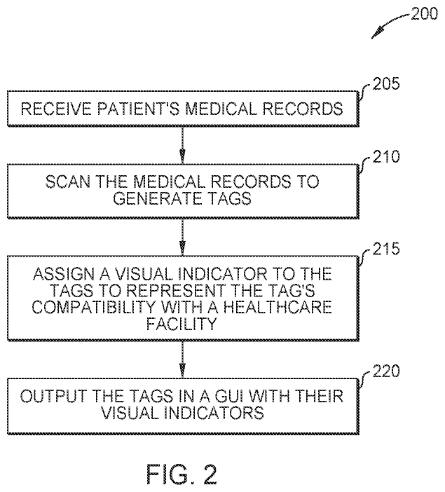

FIG. 2 is a flowchart of a method 200 for tagging a patient's medical records, according to one embodiment. At block 205, the evaluation portal (e.g., the evaluation portal 105 in FIG. 1) receives the patient's medical records. While the embodiments herein refer to a “patient”, the method 200 can be used for by any person (regardless whether they are currently admitted into a healthcare facility). For example, the method 200 can be used by a person or a legal guardian to identify a healthcare facility that is suitable to the person when that person is not currently in a healthcare facility. The person can use the method 200 to evaluate whether a particular facility, or which facility from a candidate pool of facilities, is a good fit for the person. However, for ease of explanation, the method 200 is described in the context of an evaluator (e.g., a healthcare professional or staff member) who is attempting to select a facility to transfer a current patient. The method 200 can also be used to determine that the patient should not be transferred a different facility. For example, the method 200 may indicate the patient's current facility is the best facility for the patient, and thus, she should not be transferred.

At block 210, the tag generator at the evaluation portal (e.g., the tag generator 110 in FIG. 1) scans the medical records to generate tags for the patient. In one embodiment, the tag generator performs a text search to identify relevant tags for the patient such as the patient's medical diagnosis, insurance carrier, current prescriptions, special medical needs, and the like. In one embodiment, the tag generator includes a tag database of relevant medical information which is then compared to the text in the patient's medical records. If the medical records contain words that match an entry in the tag database, the tag generator then generates a tag for the patient. For example, the tag database may list all the known medical diagnoses and their diagnosis codes. The tag generator can then parse the medical records to determine whether they contain text that matches one of the medical diagnoses or diagnosis codes stored in the database. If so, the tag generator generates a tag for the diagnosis. Similarly, the tag database may list all known drugs as well as medical treatments that do not use drugs (e.g., physical therapy treatments, surgeries, psychological treatments, etc.). The tag generator can scan the medical records to determine whether the patient is prescribed one of the drugs or is currently undergoing (or will undergo in the future) one of the medical treatments. If so, the tag generator can generate tags for those drugs and treatments.

In one embodiment, the medical record may list a medical diagnosis, drug, or treatment that does not exactly match the entry in the database. As such, the tag generator may use natural language processing (which can include the use of a ML model) to identify derivatives of the medical diagnoses, drugs, or treatments in the medical records. For example, the database may list “heart arrhythmia” but the medical records may instead recite “irregular heartbeat.” Using natural language processing, the tag generator may determine that an irregular heartbeat matches heart arrhythmia, and in response, create a heart arrhythmia tag for the patient. Similarly, the text may use the plural form of the diagnoses or prescriptions. The tag generator can use natural language processing to still match these derivatives to the information in the tag database.

In yet another example, the medical records may recite the diagnoses in a different format than they are listed in the tag database. For example, the tag database recites “heart disease” but the medical records may recite the derivative “disease of the heart”. Using natural language processing, the tag generator can identify and correlate the various different derivatives for the ailments, diagnoses, prescriptions, drugs, and medical treatments listed in the tag database with the text in the medical records.

In one embodiment, the natural language processing can include the use of ML models to correlate the information in the medical records with the information in the tag database to generate the tags. For example, the tag generator can use a ML model that is trained to recognize the various derivatives of ailments, diagnoses, prescriptions, drugs, and medical treatments. In this manner, the medical records do not have to have medical information that precisely aligns with the information listed in the tag database in order to identify and generate the tags.

At block 215, the compatibility calculator (e.g., the compatibility calculator 120 in FIG. 1) assigns a visual indicator to the tags to represent the tags' compatibility with a healthcare facility. In one embodiment, the compatibility calculator stores a list of facility attributes (e.g., the facility attributes 125 in FIG. 1) about a facility or about each facility in a pool of healthcare facilities (e.g., all the facilities in a healthcare network or all the facilities that are part of a healthcare provider). The evaluator or some other entity may provide the facility attributes to the evaluation portal before the method 200 began, or the attributes can be provided to the health portal at the medical records are received after block 205.

In one embodiment, the facility attributes indicate which ailments or diseases a healthcare facility can treat, which prescriptions the facility can administer, the medical equipment in the facility, the lab capabilities of the facility, the expertise of the staff at the facility, the insurance accepted at the facility, and the like. For example, a hospital may be able to treat almost all diseases while an assisted living facility may be able to treat a more limited set of ailments or diseases. In another example, a first facility may have medical equipment for performing a specific medical test (e.g., a CT scan) or a lab that can run a specific test in-house while a second facility cannot. Also, the facility attributes can indicate a cost-per-day and compatibility with certain types of medical payers. This information can indicate whether a specific insurance is accepted and whether there are any out-of-pocket expenses.

In one embodiment, the compatibility calculator compares the generated tags at block 210 to the attributes of one or more facilities to determine the compatibility of the patient with the facility. For example, if the tag generator identified a tag for a particular illness, the compatibility calculator can determine whether the facility attributes indicate a corresponding facility can treat that illness. In another example, if the patient has a tag indicating she needs a special lift, the compatibility calculator can determine from the attributes whether that facility has the lift. In yet another example, if the patient has a tag indicating her insurance, the compatibility calculator can determine from the attributes whether that facility accepts that insurance (e.g., whether the facility is in-network or out-of-network).

As mentioned above, while for some tags the compatibly analysis performed by the compatibility calculator may be a simple binary result—e.g., yes, the facility does have a special lift needed by the patient, or no, the facility does not have the lift—for other tags the compatibly analysis may result in a range of values. For example, the facility may accept the patient's insurance, but it may not prefer that insurance relative to other insurers. Or the facility may have the expertise for treating a particular illness, but the facility may be primary set up to handle a different type (or types) of disease. In those situations, the compatibility between one of the tags and the facility attributes may be a sliding scale ranging between completely compatible (e.g., preferred) or incompatible (cannot service the patient's need).

For example, the sliding scale may include low compatibility, medium compatibility, and high compatibility for certain tags, or assigns a numerical value (1-10). The facility attributes may associate different diagnoses, prescriptions, or medical treatments with different values of the sliding scale (e.g., a compatibility score). For instance, all diseases an ailments associated with the heart may be assigned a value of 10 indicating the facility specializes in those diseases. Thus, if the patient has a tag related to heart disease, this tag is assigned a value of 10 indicating very high compatibility. All other diseases and ailments associated with the cardiovascular system (but not specifically with the heart) may be assigned a value of 9 in the scale. All diseases and ailments associated with the respiratory system may be assigned a value of 8 in the scale, while all diseases and ailments associated with the musculoskeletal system and the nervous system are assigned a value of 7 in the scale, and so forth. In this manner, the facility attributes can rank diseases and ailments which then can be correlated to the tags to determine the compatibility of the patient with the facility. This ranking can also be applied to other types of attributes such as prescriptions and medical treatments.

After determining the compatibility between the tags of a patient and the facility using the facility attributes, the compatibility calculator assigns a visual indicator to the tags to represent their individual compatibility scores with the facility. For example, if the tag indicates the patient needs a lift, and the facility has the lift, the tag may be assigned the color green; but if not, the tag may be assigned the color red. For tags where their compatibility scores are assigned using a range of values (e.g., somewhere between completely compatible and incompatible or a numerical range), the tags may be assigned a corresponding range of colors or different shades of the same color. For example, if a tag indicates the patient needs frequent CT scans but the facility offers CT scans on a limited time schedule or has a limited number of CT machines, this tag may be assigned the color yellow indicated the facility is not completely incompatible with the tag (i.e., the patient's need) but also might not be the best fit relative to a facility that provides greater access to CT scans. Or if the tag indicates the patient has an insurance provider that is accepted, but not preferred by the facility, or the patient may have some out-of-pocket expenses, the tag may be assigned the color yellow.

In another example, the compatibility calculator can assign a shade to a range of values of the compatibility score assigned to a tag. If the score is assigned a compatibility score from 1-10, each value can be assigned a different shade between the colors black and white. The value 10, which represents high compatibility can be assigned white while the value 1 which represents low compatibility can be assigned black. The values 2-9 can then be assigned progressively lighter shades.

As another example, the compatibility calculator can use a numerical value as the visual indicator for the tags. For example, if the tag is assigned a compatibility score along a sliding scale, the compatibility calculator may display the score (e.g., a value from 1-10) as the visual indicator.

However, assigning colors to the tags is just one example of a visual indicator. In other embodiment, the tags may be assigned different luminance values or different sizes in response to their compatibility scores. For example, a brighter luminance value indicates greater compatibility while a dimmer luminance indicates lower compatibility, or larger/bolder text in the tag indicates greater compatibility while a smaller/less bold text in the tag indicates lower compatibility.

In another embodiment, different highlighting or visual effects (e.g., blinking) can be applied to the tags to represent their compatibility with a particular facility. The tags can blink or flash where a faster blinking rate indicates greater compatibility while a slower blinking rate indicates lower compatibility.

In one embodiment, assigning visual indicators to the tags in response to their compatibility with a particular facility can result in the adjusted tags 130 illustrated in FIG. 1. Thus, the adjusted tags can include text indicating medical information regarding the patient or person as well as a visual indicator for the tag to indicate the compatibility between that tag and a particular healthcare facility.

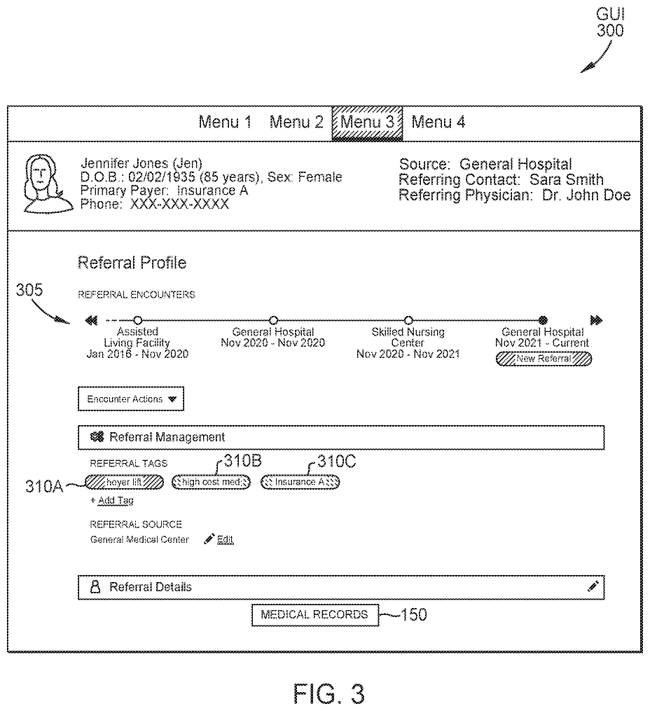

At block 220, the GUI creator outputs (or transmits for display) the tags in a GUI with their visual indicators. For example, the tags can be listed at or near the top of the GUI where they are most likely to be seen by the evaluator. Further, the GUI can include the patient's medical records which formed the basis for the tags. For example, the medical records can be at the bottom of the GUI in textual form, or the GUI may include links for downloading a copy of the medical records—e.g., a text document or a PDF of the medical records. The details of the GUI are discussed below in FIG. 3.

In one embodiment, the evaluation portal includes an authentication system for ensuring only authorized individuals can access the GUI. Since the GUI can include sensitive or private health information such as the tags and the medical records, the evaluation portal can secure this information by ensuring any person wanting to view the GUI has been authorized. In one example, the evaluation portal may make the GUI accessible using a web browser, but the evaluator must provide security credentials to the web browser before the GUI is displayed. In another embodiment, the evaluation portal may be a secure application running locally on the evaluator's computer device. The evaluation portal may still require security credentials or biometric information (e.g., a face scan) before displaying the GUI to ensure it is the evaluator rather than a nefarious user who is attempting to access the GUI. This authorization may occur before the GUI is outputted for display at block 220.

In one embodiment, the method 200 can be used as part of a prophylaxis treatment or to make a medical diagnosis. As an example of a prophylaxis treatment, the patient may have recently undergone a surgery or procedure that is known to cause serious side effects or additional complications if the patient's recovery is not carefully monitored. In that case, the evaluation portal can use the method 200 to identify, using the tags, a healthcare facility that specializes in this type of recovery or has all the equipment and expertise necessary to help the patient recover and avoid the side effects or complications.

As another example of prophylaxis treatment, the patient may have family history of a particular disease but has not yet been diagnosed with that disease. This family history may have been noted in the patient's medical record and then tagged by the tag evaluator at block 210. The tag can then be used to identify a healthcare facility that specializes in the disease (and its diagnosis) even though the patient has not yet been diagnosed with that disease. This may increase the likelihood that if the disease manifests itself in the patient while at the specialized healthcare facility, it will be detected earlier, and thus increase the likelihood it can be treated effectively.

As an example of using the method 200 to perform a medical diagnosis, the patient may be at a current facility where the doctors are unsure of the diagnosis. Instead, the medical record may indicate some potential candidate diagnoses (e.g., a potential neurological disorder). These candidate diagnoses can be tagged by the tag evaluator and then compared to the attributes or one or more healthcare facilities as discussed at block 215. The evaluator can then recommend transferring the patient to a healthcare facility that specializes in the type of disorder the doctors at the current facility believe the patient has. This can help the patient receive a correct diagnosis.

As another example of using the method 200 to perform a medical diagnosis, the patient may be at a current facility that lacks the testing equipment or lab capabilities to run sufficient tests to generate a diagnosis. The doctors may update the medical record to indicator which types of tests are needed to diagnosis the patient. When executing the method 200, the evaluator portal can then tag these test which are then compared to the facility attributes of one or more healthcare facilities to determine whether they can perform those test. In this manner, the method 200 can be used to transfer the patient to a healthcare facility with the necessary infrastructure or expertise to provide a diagnosis.

FIG. 3 is a GUI 300 displaying tags corresponding to a patient, according to one embodiment. In one embodiment, the GUI 300 is created using the method 200 in FIG. 2. The GUI 300 can be output on a web browser, or can be displayed by an application executing on a user device.

The top of the GUI 300 describes a patient (e.g., a referral) such as date of birth (DOB), insurance (“Insurance A”), contact information, current facility (e.g., General Hospital in this example), the referring contact, referring physician, and the like. As discussed above, the GUI 300 may be generated in response to an evaluator receiving a request to transfer the patient from their current facility. For example, the patient may have recently completed surgery at the General Hospital and should be moved to a recovery center or an assisted living facility before being released. But this is just one event that might cause a patient to be transferred. Other events include the doctors at the current hospital being unable to identify a diagnosis, the patient needs an operation or medical treatment that the current facility does not provide (or does not provide well), or the patient may want to be transferred to be closer to a relative or family friend. Further, the GUI 300 can be used to for a referral who is not currently admitted to any healthcare facility.

The GUI 300 also includes a timeline 305 indicating referral encounters where the patient/referral was previously admitted. In this case, the patient was in an assisted living facility before being transferred to General Hospital and then transferred to a skilled nursing center. However, the patient was then transferred back to the General Hospital and is now looking to be transferred to a new facility. For example, while at the skilled nursing center, the patient may have become sick or needed a surgery that could only be provided at the hospital. Now that the surgery or treatment has been performed, the patient no longer needs to stay at the hospital but can be moved to a different facility that may be able to help her recover and be less expensive.

The GUI 300 includes tags 310 which are generated using the method 200. Specifically, the tag generator can parse the medical records 150 (which are also included in the GUI 300) to identify the tags as discussed in the method 200. Further, these tags can be assigned visual indicators to indicate their compatibility with a particular facility. In this example, the tags 310 are compared to the attributes of the referral source—i.e., the General Medical Center. Thus, the visual indicators assigned to the tags 310 indicate the compatibility of the tags 310 of the patient and the General Medical Center. In this example, the tags 310 have different hatching to indicate different visual indicators. Here, the tags 310B and 310C have the same hatching (or visual indicator) representing that they have the same compatibility with the General Medical Center. However, the tag 310A has a different hatching (or visual indicator) indicating it has a different compatibility with the General Medical Center than the tags 310B and 310C.

For example, the tag 310A (“Hoyer lift”) indicates the patient needs this special lift, but the visual indicator may indicate the General Medical Center does not have that lift. Thus, the visual indicator may be red indicating they are incompatible. In contrast, the tag 310B (“high cost med”) and tag 310C (“Insurance A”) may be assigned visual indicators indicating the General Medical Center is compatible with those tags. For example, the tags 310B and 310C may be colored green to indicate they are compatible with the General Medical Center.

The GUI 300 also includes an “Add Tag” feature so that the evaluator can add a tag to the GUI 300. For example, after reviewing the medical records 150, the evaluator may identify a tag that was not identified by the tag generator. The tag generator may have missed the tag because it was stated in a confusing way. For example, the medical records 150 may indicate a diagnosis using a non-standardized abbreviation “On November 15, Jennifer Jones was diagnosed with Prinz Ang” which the evaluator may recognize as Prinzmetal Angina. Or the medical records may include handwritten notes that the tag generator was unable to parse, but the evaluator was able to read to identify a tag. Once the tag is entered, in the background, the evaluator tag can perform blocks 215 and 220 in method 200 to refresh the GUI 300 so it now has the new tag along with a corresponding visual indicator representing its compatibility with the healthcare facility—i.e., the General Medical Center.

In one embodiment, the evaluator can change the healthcare facility being evaluated as a transfer candidate. For example, the evaluator can click or touch the “edit” link next to General Medical Center that the opens a pop-up window or takes the user to a different GUI where the evaluator can select a different healthcare facility. Once selected, the evaluator portal can update the GUI 300 to change the visual indicators for the tags 310 to indicate their compatibility with the new healthcare facility. That is, the evaluator portal would not need to update the text in the tags 310 (or look for different tags) since the same patient/referral is being considered, but does update the visual indicators assigned to the tags 310. For example, the new facility may have a Hoyer lift, in which case the visual indicator for the tag 310A is changed to indicate this tag is compatible with the new healthcare facility. In addition, the visual indicators for the tags 3106 and 310C may also change, or they could also stay the same depending on the attributes of the new healthcare facility.

In one embodiment, different portions of the GUI 300 can be minimized or maximized. For example, when the GUI 300 is initially displayed, the patient information, timeline 305, and the tags 310 may be shown in the GUI 300 while the medical records 150 are minimized or hidden. However, if the evaluator wants to review the medical records 150, the evaluator can expand the portion of the GUI 300 containing the records 150 and minimize some or all of the portion of the GUI 300 displaying the patient information, timeline 305, and the tags 310.

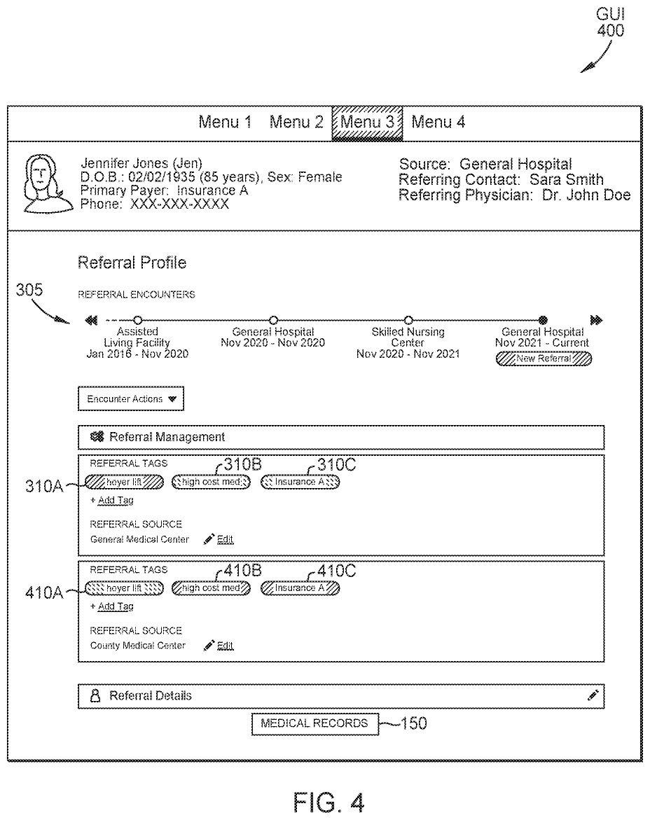

FIG. 4 is a GUI 400 displaying tags corresponding to a patient, according to one embodiment. In one embodiment, the GUI 400 is created using the method 200 in FIG. 2. The GUI 400 can be output on a web browser, or can be displayed by an application executing on a user device.

The GUI 400 is the same as the GUI 300 except that the Referral Management section has been expanded to show comparing the same tags to different healthcare facilities. That is, while the GUI 300 illustrates comparing the tags 310 to one healthcare facility at a time, in GUI 400 the tags are compared to different healthcare facilities at the same time.

The GUI 400 displays different sets of the tags—i.e., tags 310A-C and tags 410A-C, each set being assigned visual indicators for a different healthcare facility—i.e., the General Medical Center and the County Medical Center. Instead of the evaluator having to switch between healthcare facilities in GUI 300, the GUI 400 displays different sets of the tags where the visual indicators for each set are compared to the attributes of a different healthcare facility. For example, the GUI 400 includes two sets of the tags displayed on different rows. The first row of tags 310A-C have visual indicators representing their compatibility with the General Medical Center but the second row of tags 410A-C have visual indicators representing their compatibility with the Country Medical Center. Specifically, while the tags 310 and 410 have the same text (i.e., the tags 310A and 410A both correspond to a “Hoyer lift”, the tags 310B and 410B both correspond to “high cost med”, and the tags 310C and 410C both correspond to “Insurance A”) the tags have different hatching. That is, the hatching for tag 310A is different from tag 410A, the hatching for tag 310B is different from tag 410B, and so forth. This may indicate the tag 310A is less compatible with the General Medical Center while the tag 410A is more compatible with the County Medical Center and the tags 310B and 310C are more compatible with the General Medical Center while the tags 4106 and 410C is less compatible with the County Medical Center. In this manner, the underlying text of labels for the set of tags is the same, but the visual indicators can change depending on the compatibility of the tags with the attributes of the particular healthcare facility. Of course, for some of the tags, the visual indicators may be the same in different sets of tags. For example, if the General Medical Center and the County Medical Center both had Hoyer lifts, then the visual indicators for the tag 310A and the tag 410A may be the same (e.g., both be green).

While the GUI 400 shows displaying a set of tags for two healthcare facilities, the GUI 400 can be expanded to display a set of tags for any number of healthcare facilities. In this manner, the evaluator can quickly evaluate multiple candidate healthcare facilities for the patient/referral using the same instance of the GUI 400.

In another embodiment, the evaluator portal may first compare the tags to the attributes of multiple healthcare facility, but then display in the GUI 400 the tags (and their visual indicators) for the top three or top five healthcare facilities. For example, the healthcare provider may have twenty healthcare facilities. The compatibility calculator can then compare the tags of the patient to the facility attributes for all twenty of the healthcare facilities. The compatibility calculator can then use the resulting compatibility scores for the tags 310 to identify the top two or top three healthcare facilities. For example, the compatibility calculator may average the compatibility scores of the tags, or sum the compatibility scores to identify the healthcare facilities that are the most compatible with the tags. In another embodiment, the compatibility calculator can require the healthcare facilities to have a minimum average or minimum total compatibility score before they can be considered as a top healthcare facility. For example, to be displayed, a healthcare facility must have a minimum compatibility score above X and be one of the five healthcare facilities with the highest compatibility score. Once the top healthcare facilities are identified, the evaluator portal can then display a set of the tags (e.g., the tags 310A-C and the tags 410A-C) for each of the top healthcare facilities, with their corresponding visual indicators, in the GUI 400. In this manner, the GUI 400 can display multiples sets of tags each with visual indicators for a different healthcare facility.

Moreover, the GUI creator can use the total compatibility scores or the average compatibility scores of the tags to arrange the sets of tags. For example, the GUI creator may list first in the GUI 400 the healthcare facility with the greatest compatibility with the tags (e.g., the highest total compatibility score or the greatest average compatibility score). Thus, the cumulative scores corresponding to the healthcare facilities can be used to rank the healthcare facilities when being displayed in the GUI 400.

Like above, different portions of the GUI 400 can be minimized or maximized. For example, when the GUI 400 is initially displayed, the patient information, timeline 305, and the tags 310, 410 may be shown in the GUI 400 while the medical records 150 are minimized or hidden. However, if the evaluator wants to review the medical records 150, the evaluator can expand the portion of the GUI 400 containing the records 150 and minimize some or all of the portion of the GUI 400 displaying the patient information, timeline 305, and the tags 310, 410.

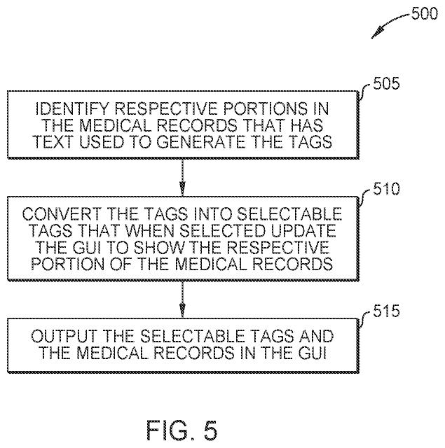

FIG. 5 is a flowchart of a method 500 for generating selectable tags for a GUI, according to one embodiment. At block 505, the tag generator (e.g., the tag generator 110 in FIG. 1) identifies respective portions in the patient's medical records that have text used to generate the tags. That is, when the tag generator scans the patient's medical records as described in block 210 of the method 200, the tag generator can identify or track the portions of the medical record that were used to generate the tag. For example, the tag generator may have a data structure that identifies a location of the text in the medical record used to generate a tag, or the tag generator may directly save the text used to derive a tag into the data structure. In this manner, the tag generator can separately track or identify the text used to generate the tags from the remaining text in the medical records.

At block 510, the tag generator converts the tags into selectable tags that, when selected, update the GUI to show the respective portion of the medical record that was used to generate the tag. Referring to the GUI 300 in FIG. 3, the tags 310 may be selectable such that when the evaluator clicks or touches the tags 310, the GUI is updated to display the portion of the medical records 150 that generated the tag. For example, the GUI creator refreshes the GUI 300 so that the Referral Details section displays the text used to generate the selected tag.

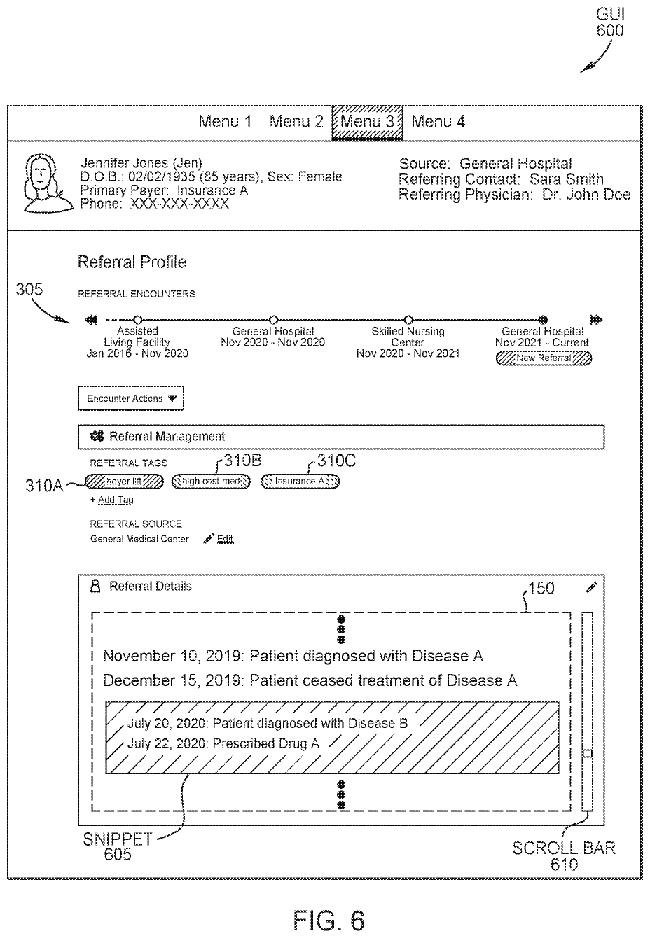

In one embodiment, the evaluator portal updates the GUI 300 by displaying the portion of the text corresponding to the selected tag 310 in the Referral Details section by automatically scrolling to a location in this section containing the relevant text. That is, the evaluator portal can identify the location using the data structure maintained by the tag generator and adjust the Referral Details section so the text is displayed. This is described in more detail in FIG. 6.

In another embodiment, when a tag 310 is selected, the evaluator portal opens up a pop-up window that displays the text corresponding to the selected tag 310. That is, the evaluator portal can generate a separate GUI that is displayed over the GUI 300 that has the text. In addition to displaying the text used to generate the tag, to provide additional context, the evaluator portal can also display a few sentences or lines above and below the relevant text in the pop-up window which were not used to generate the tag but may nonetheless be related. For example, the sentences before or after the relevant text may indicate previous diagnoses or medications for the patient.

At block 515, the evaluator portal outputs the selectable tags and the medical records in the GUI. The evaluator can then select that tags which updates the GUI or opens up a pop-up window to display the text used to generate the selected tag.

FIG. 6 is a GUI 600 displaying text in a medical record used to generate a tag, according to one embodiment. The GUI 600 illustrates one example of how the GUI 300 in FIG. 3 can be updated or refreshed when the evaluator selects one of the tags 310. The GUI 600 assumes that the evaluator has selected (e.g., clicked or touched) the tag 310B. In response, the evaluator portal outputs the GUI 600 which is the same as the GUI 300 except that the Referral Details section has been updated to display the snippet 605 used to generate the tag 310B. For example, the evaluator may want additional details on what specifically is the high cost medication referenced in the tag 310B. As such, the evaluator can select the tag 310B which causes the evaluator portal to display the GUI 600 where the snippet 605 is displayed.

As shown, the snippet 605 includes a portion of the patient's medical record 150 where the patient was diagnosed with Disease B and then prescribed Drug A. The tag generator, when scanning the medical records 150, recognized that Drug A was a high cost medication, and in response, generated the tag 310B.

In one embodiment, the tag generator also searched the remainder of the medical records 150 to ensure there is no entry indicating the prescription for Drug A has expired. As a result, the tag generator may know the patient is still regularly taking the Drug A, and thus, the tag 3106 should be generated. In contrast, the tag generator did not create a tag for Disease A since the medical history includes an entry indicating the patient was diagnosed with Disease A, but also has an entry indicating the patient is no longer being treated for that Disease A. Thus, the tag generator did not generate a tag for Disease A, or a tag indicating the patient is undergoing any medical treatments related to Disease A.

In one embodiment, the evaluator portal displays the snippet 605 in the Referral Details by automatically scrolling to the portion of the medical records 150 containing the snippet 605. For example, the evaluator portal may use the scroll bar 610 to scroll to the portion of the records 150 that contains the snippet 605.

In addition to displaying the snippet 605, in this example the GUI 600 displays the text immediately preceding the snippet 605 regarding the diagnosis of Disease A. The GUI 600 may also display text in the record 150 that is after the snippet 605. Doing so may provide context to the evaluator, even though this text was not used to generate the selected tag 310B.

Further, the GUI 600 includes a visual indicator for the snippet 605 which is shown using the hatching, but this is not a requirement. The visual indicator can be similar to the visual indicators for the tags 310, or could be different. For example, the GUI 600 can bold the text of the snippet 605 relative to the other text in the medical records 150, or color or highlight the text in the snippet 605. In another example, the GUI 600 can outline the snippet 605 using a colored box. In any case, the visual indicator enables the evaluator to quickly distinguish between the text in the snippet 605 used to generate the selected tag 3106 from the other text in the medical records 150 being displayed in the Referral Details section.

As mentioned above, updating the Referral Details section to display the snippet 605 is just one example of displaying the text used to generate one of the tags 310. In other embodiments, the snippet 605 (and some of the entries or text around the snippet 605) can be displayed in a pop-up window—e.g., a separate GUI.

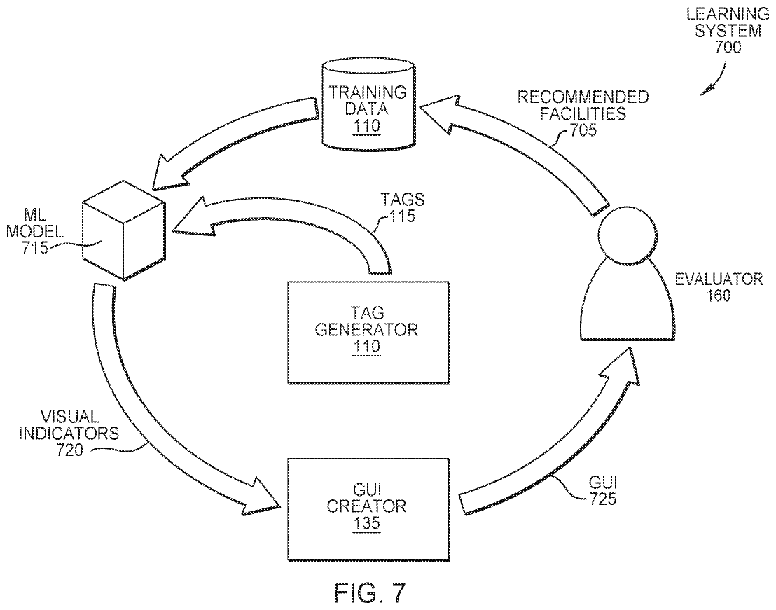

FIG. 7 illustrates a learning system 700 for assigning visual indicators to tags, according to one embodiment. The learning system 700 includes a ML model 715 which is trained using training data 710 generated from the evaluators 160 recommendations or selections. As described above, the evaluator 160 can use the tags displayed in the GUIs to decide whether or not to recommend moving a patient to a particular healthcare facility. Using the GUI 400 as an example, the evaluator 160 can choose whether to transfer the patient to the General Medical Center or the County Medical Center. The evaluator 160 can inform the evaluator portal of this choice.

By knowing the evaluator's 160 recommendation, the evaluator portal can generate the training data 710 to then train the ML model 715. For example, the training data 710 can include the tags and their visual indicators assigned by the compatibility calculator using the facility attributes. However, the evaluator 160 may have chosen a first facility for the patient that had relative lower compatibility scores for the patient's tags than a second facility. For example, for the first facility, the compatibility calculator may have assigned a high compatibility score to a first tag, but low compatibility scores to all the other tags. In contrast, for the second facility, the compatibility calculator may have assigned a low compatibility score to a first tag, but high compatibility scores to all the other tags. Thus, the overall compatibility score for the first facility is lower than the overall score for the second facility, all things being equal. However, the evaluator 160 may have recommended the first facility because the first tag is more important than the other tags. As discussed below, the learning system 700 can learn the preferences of the evaluator 160 over time to train the ML model 715. The ML model 715 can then be used to adjust (or set) the visual indicators to the tags to better match the preferences of the evaluator 160.

In one embodiment, the training data 710 lists the tags of the facilities that were recommended (or selected) by the evaluator 160 as well as the tags of the facilities that were not recommended. That is, the training data 710 can be derived from recommend facilities 705 which link the tags to the facility that was selected or recommended for the patient. This can implicitly indicate which facilities where not selected. Again referring to GUI 400, by knowing the evaluator recommended the patent be transferred to the General Medical Center and not the County Medical Center, the training data 710 can reflect that the tags 310 were used to recommend the General Medical Center but not the Country Medical Center.

The training data 710 can be gathered over a set time period (e.g., a month) and can be based on recommendations made from one, or multiple, evaluators 160. The training data 710 can be referred to as annotated training data since it includes the “answer” indicating whether a facility was recommended or not.

As shown, the training data 710 is used to train the ML model 715. The ML model 715 can then learn relationships between the tags and the different healthcare facilities. For example, the ML model 715 can learn that when a particular tag is generated, that patient is almost always referred to a particular facility. Further, the ML model 715 can learn that one tag (or tags) are much more important or critical than others. For example, when a tag has a low compatibility for a particular healthcare facility, that facility is almost never selected. The ML model 715 can also learn relationship between the tags. For example, when a patient has a certain group of tags, the evaluator typically recommends that patient be transferred to a particular healthcare facility. These are just some of the examples of where a ML model 715 can be trained using training data 710 reflecting the preferences of the evaluator 160.

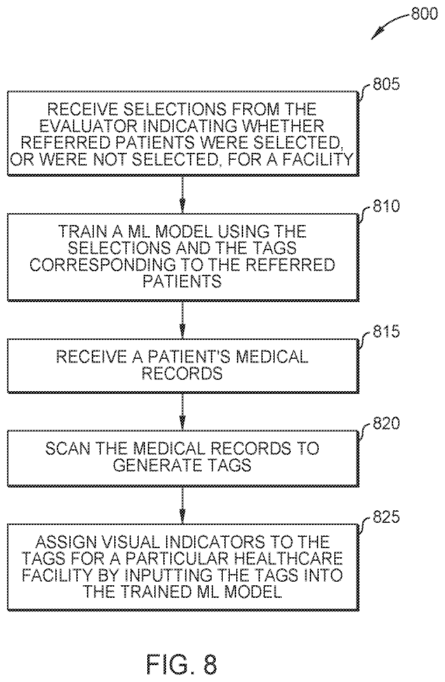

Once trained, the ML model 715 can then be used to assign or adjust the visual indicators of the tags. When a new referral process is begun, the tag generator 110 can forward the tags 115 to the trained ML model 715 as inputs. The ML model 715 can then evaluate the tags and output visual indicators 720 for those tags. In one embodiment, the ML model 715 may be solely used to assign the visual indicators 720 to the tags 115. However, in another embodiment, the learning system 700 may use both the ML model 715 and the compatibility calculator to assign the visual indicators 720 to the tags 115. For example, the learning system 700 may first use the compatibility calculator to assign the visual indicators by comparing them to the facility attributes to generate compatibility scores. The learning system 700 can then use the ML model to adjust those compatibility scores before assigning the visual indicators 720. In yet another embodiment, the compatibility calculator and the ML model 715 can generate independent compatibility scores which are then weighted to determine the visual indicators 720. This is discussed in more detail in the flowchart for FIG. 8.

The visual indicators 720 are then sent to the GUI creator 135 where it outputs the GUI 725 that is then viewed by the evaluator 160. In this manner, the learning system 700 provides a feedback loop where the evaluator's recommendations can be used to train a ML model 715 so that future tags provided by the system 700 have visual indicators 720 that reflect the evaluator's own preferences.

FIG. 8 is a flowchart of a method 800 for assigning visual indicators to tags corresponding to a patient using a ML model, according to one embodiment. At block 805, the evaluator portal receives selections from the evaluator indicating whether patients were selected, or were not selected, by the evaluator for a facility. For example, the method 800 may occur after the method 200 has completed and the evaluator has used a GUI to select (or recommend) a patient to be transferred to a particular facility. This selection (or recommendation) is then fed back to the evaluator portal so it knows the evaluator's choice. Thus, the evaluator portal knows the patient's tags, the compatibility score and visual indicators assigned to those tags, and which facility the evaluator decided was the best fit for the patient based on those tags. This implicitly informs the evaluator portal which facilities the evaluator did not think were the best fit for the patient.

The evaluator portal may wait until it receives a certain amount of selections from the evaluator (or evaluators) before proceeding. For example, the method 800 may wait until hundred, or thousands of selections from the evaluator are received before training the ML model.

At block 810, the evaluator portal trains the ML module using the selections and the tags corresponding to the referred patients. That is, the evaluator portal may generate training data using the tags, their attributes, and the recommended facility for each selection received from the evaluator. This training data can then be used to train the ML model. The embodiments herein are not limited to any particular type of ML model, but can include various types of neural networks.

Using the training data, the ML model can learn the preferences of the evaluator. For instance, the ML model can learn relationships between certain tags and particular facilities, relationships between a group of tags and a particular facility, and relationships between the tags themselves (e.g., some tags are more critical than others).

For example, the ML model can learn that when a patient has a certain tag, the patient is almost always assigned to a particular facility, regardless whether the compatibility calculator assigned a low or high compatibility score to the tag. For example, the facility attribute may indicate the healthcare facility accepts the patient's insurance but it is less preferred. Thus, the compatibility score between a tag the insurance and the facility may have a low compatibility score. However, that facility might be the only facility that accepts that insurance, so the evaluator almost always recommends the patient be transferred to the facility. The ML model can then learn this relationship between the particular tag and facility which is not reflected in the facility attributes.

As an example of learning relationships between a group of tags and a particular facility, the ML model can learn that the evaluator recommends a particular facility when a patient has a certain group of tags, regardless of their compatibility scores. For example, some of the tags in the group may have very low compatibility scores, but nonetheless, when a patient has that group of tags, a particular facility is typically recommended by the evaluator.

As an example of learning relationships between the tags themselves, the ML model can learn from the training data which types of tags are more critical than others. For example, the ML model may learn that when the patient has a tag related to special medical equipment, the capability scores of those tags primarily determine whether a facility is selected. In contrast, the evaluator almost never recommends a facility that has a low capability score with those types of tags.

As discussed in more detail below, these relationship learned by the ML reflect the evaluator's preferences and can be used to adjust or set the visual indicators assigned to the tags.

At block 815, the evaluator portal receives medical records for a new patient (or an old patient who is being transferred again). This can be performed using any of the techniques described at block 205 of method 200.

At block 820, the evaluator portal scans the medical records to generate tags. This can be performed using any of the techniques described at block 210 of method 200.

At block 825, the evaluator portal assigns visual indicators to the tags for a particular facility by inputting the tags into the trained ML model. For example, the ML model may accept as inputs the tags and one of the facilities from the pool of candidate healthcare facilities. Block 825 can be performed for a particular facility selected by the evaluator or can be repeated for each facility in the pool health facilities to generate a set of tags for each facility, where each set of tags can have different visual indicators as shown in GUI 400.

Referring to the example relationships described above, the ML model can set or adjust the compatibility scores for the tags which in turn are used to set the visual indicators. For example, if the ML model learned that when a patient has a particular tag, regardless of the compatibility score for that tag, that patient is almost always referred to a particular facility, the ML model will set a high compatibility score for that tag for that particular facility but set a low compatibility score for all other facilities.

As another example, if the ML model learned a relationship between a group of tags and a particular facility, the ML model will set a high compatibility score for those tags for that particular facility but set a low compatibility score for those tag for all other facilities.

As another example, if the ML model learned which types of tags are more critical than others, it may be assigned the highest compatibility score to ensure it sticks out visually from all the other tags. Or the ML model may assign those types of tags a different type of visual indicator from the other tags. For example, these types of tags may flash when displayed in the GUI while the other types of tags are simply assigned a color.

In one embodiment, the output of the ML model is used to set the compatibility score, and thus, the visual indicator of the tag. For example, when the evaluator portal first begins operating, it can rely on the compatibility calculator to set the compatibility scores and the visual indicators. However, after using the method 800 to receive feedback from the evaluator and train the ML model in response to the evaluator's preferences, the evaluator portal can switch to using the compatibility scores generating by the ML model to set the visual indicators without using the compatibility calculator.

In another embodiment, after the ML Model is trained, the evaluator portal can use both the compatibility scores generated by the ML model and the compatibility scores generated by the compatibility calculator to set the visual indicators. For example, the compatibility scores generated by the ML model can be used to adjust the initial compatibility scores generated by the compatibility calculator. The visual indicators can then be set by the combined compatibility scores. In another example, the evaluator portal can use weights to combine the compatibility scores generated by the ML model and the compatibility calculator and then set the visual indicators using the weighted compatibility score.

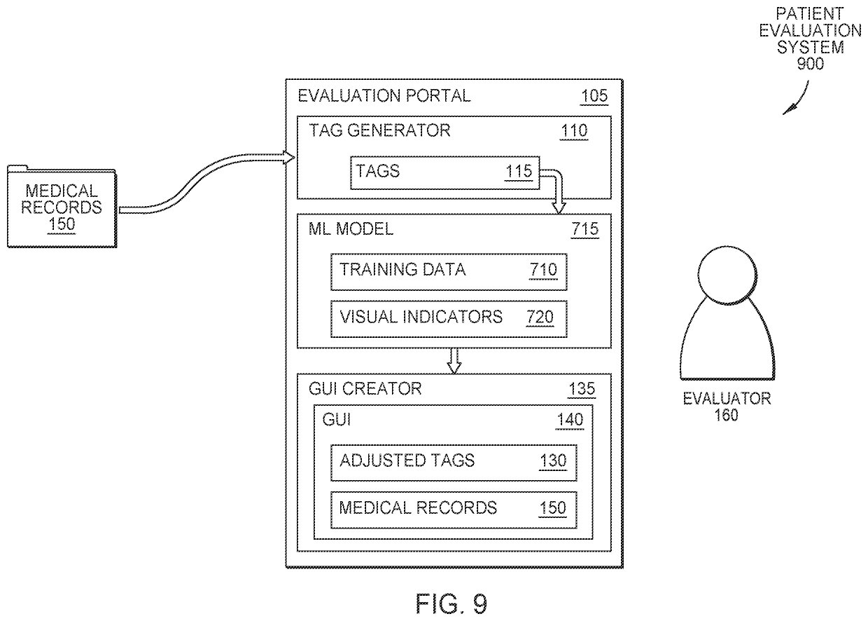

FIG. 9 illustrates a block diagram of a patient evaluation system 900 for providing tags for a patient, according to one embodiment. In this embodiment, the compatibility calculator 120 in FIG. 1 is replaced by the ML model 715. That is, FIG. 9 illustrates that the system 900 can assign visual indicators to the tags using just the ML model 715. For example, FIG. 1 may represent the patient evaluation system 100 at start up when training data for the ML model 715 is being collected. However, once the ML model 715 is trained, the system can switch to the state illustrated in FIG. 9 where the compatibility calculator 120 is deactivated and the ML model 715 is solely used to assign the visual indicators 720 to the tags 115. The visual indicators 720 determined by the ML model 715 are then used by the GUI creator 135 which generating the GUI 140.

Alternatively, as discussed above, in some embodiments both the compatibility calculator 120 and the ML model 715 are used to assign the compatibility scores and the visual indicators to the tag. In that case, the system 900 would still include the compatibility calculator 120.

### Example Computing Hardware

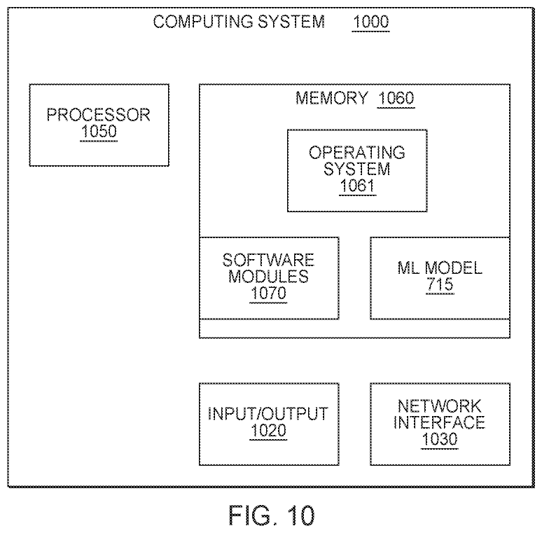

FIG. 10 illustrates a computing system 1000, which may be used to implement the evaluation portal 105 in FIGS. 1 and 9 (e.g., a computer, a laptop, a tablet, a smartphone, web server, data center, cloud computing environment, etc.), or any other computing device described in the present disclosure. As shown, the computing system 1000 includes, without limitation, a processor 1050 (e.g., a central processing unit), a network interface 1030, and memory 1060. The computing system 1000 may also include an I/O device interface connecting I/O devices 1020 (e.g., keyboard, display and mouse devices) to the computing system 1000.

The processor 1050 retrieves and executes programming instructions stored in the memory 1060 (e.g., a computer readable medium). Similarly, the processor 1050 stores and retrieves application data residing in the memory 1060. An interconnect facilitates transmission, such as of programming instructions and application data, between the processor 1050, I/O device interface, storage, network interface 1030, and memory 1060. The processor 1050 is included to be representative of a single processor, multiple processors, a single processor having multiple processing cores, and the like. And the memory 1060 is generally included to be representative of volatile and non-volatile memory elements. For example, the memory 1060 can include random access memory and a disk drive storage device. Although shown as a single unit, the memory 1060 may be a combination of fixed and/or removable storage devices, such as magnetic disk drives, flash drives, removable memory cards or optical storage, network attached storage (NAS), or a storage area-network (SAN). The storage may include both local storage devices and remote storage devices accessible via the network interface 1030. One or more ML models 715 are maintained in the memory 1060 to provide compatibility scores for the tags. Additionally, one or more software modules 1070 may be maintained in the memory to match perform the functions of the tag generator 110, compatibility calculator 120, and the GUI creator 135 discussed above.

Further, the computing system 1000 is included to be representative of a physical computing system as well as virtual machine instances hosted on a set of underlying physical computing systems. Further still, although shown as a single computing device, one of ordinary skill in the art will recognize that the components of the computing system 1000 shown in FIG. 10 may be distributed across multiple computing systems connected by a data communications network.

As shown, the memory 1060 includes an operating system 1061. The operating system 1061 may facilitate receiving input from and providing output to various components. For example, the network interface 1030 can be used to output the GUIs to display the tags along with their visual indicators. Further, the network interface can be used to receive the evaluator's selections in order to train the ML model 715.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

The following clauses describe various embodiments of the present disclosure.

Clause 1: A method, comprising: receiving a medical record for a person; scanning the medical record to generate tags, the tags providing information about at least one of medical needs or a responsible payer of the person; assigning visual indicators to the tags to represent a compatibility of the information in the tags with a particular healthcare facility; and transmitting, for display, a GUI comprising (i) the tags along with their visual indicators and (ii) at least a portion of the medical record.

Clause 2: In addition to the method of clause 1, wherein assigning the visual indicators to the tags comprises: comparing the information in the tags to facility attributes corresponding to the particular healthcare facility, the facility attributes indicating capabilities or preferences of the particular healthcare facility.

Clause 3: In addition to the method of clause 2, wherein the compatibility represented by the visual indicators is defined by whether the particular healthcare facility can satisfy the medical needs of the person based on the capabilities or preferences of the particular healthcare facility.

Clause 4: In addition to the method of clauses 1, 2, or 3, wherein scanning the medical record to generate tags further comprises: evaluating the medical records using natural language processing or a keyword search to identify text in the medical records that matches, or is a derivative of, a tag stored in a predefined tag database.

Clause 5: In addition to the method of clauses 1, 2, 3, or 4, further comprising: identifying respective portions of the medical record that has text used to generate each of the tags; and converting the tags into selectable tags that, when displayed in the GUI and selected by a user, update the GUI to show the respective portion of the medical record corresponding to a selected one of the tags.

Clause 6: In addition to the method of clause 5, wherein updating the GUI to show the respective portion of the medical record corresponding to the selected one of the tags further comprises at least one of: auto-scrolling the GUI to a portion that includes the respective portion of the medical record; or outputting for display a pop-up window containing the respective portion of the medical record.

Clause 7: In addition to the method of clauses 1, 2, 3, 4, 5, or 6, further comprising: receiving a selection from a user indicating whether the person was recommend to be transferred to the particular healthcare facility; training a machine learning model using the selection and the tags; receiving a second medical record for a second person; scanning the second medical record to generate second tags; and assigning visual indicators to the second tags based on inputting the second tags into the trained machine learning model.

Clause 8: A computing system, comprising: a processor; and memory storing an application which, when executed by the processor, performs an operation, the operation comprising: receiving a medical record for a person; scanning the medical record to generate tags, the tags providing information about at least one of medical needs or a responsible payer of the person; assigning visual indicators to the tags to represent a compatibility of the information in the tags with a particular healthcare facility; and transmitting, for display, a GUI comprising (i) the tags along with their visual indicators and (ii) at least a portion of the medical record.

Clause 9: In addition to the computing system of clause 8, wherein assigning the visual indicators to the tags comprises: comparing the information in the tags to facility attributes corresponding to the particular healthcare facility, the facility attributes indicating capabilities or preferences of the particular healthcare facility.

Clause 10: In addition to the computing system of clause 9, wherein the compatibility represented by the visual indicators is defined by whether the particular healthcare facility can satisfy the medical needs of the person based on capabilities or preferences of the particular healthcare facility.

Clause 11: In addition to the computing system of clauses 8, 9, or 10, wherein scanning the medical record to generate tags further comprises: evaluating the medical records using natural language processing or a keyword search to identify text in the medical records that matches, or is a derivative of, a tag stored in a predefined tag database.

Clause 12: In addition to the computing system of clauses 8, 9, 10, or 11, where the operation further comprises: identifying respective portions of the medical record that has text used to generate each of the tags; and converting the tags into selectable tags that, when displayed in the GUI and selected by a user, update the GUI to show the respective portion of the medical record corresponding to a selected one of the tags.

Clause 13: In addition to the computing system of clause 12, wherein updating the GUI to show the respective portion of the medical record corresponding to the selected one of the tags further comprises at least one of: auto-scrolling the GUI to a portion that includes the respective portion of the medical record; or outputting for display a pop-up window containing the respective portion of the medical record.

Clause 14: In addition to the computing system of clauses 8, 9, 10, 11, 12, or 13, wherein the operation further comprises: receiving a selection from a user indicating whether the person was recommend to be transferred to the particular healthcare facility; training a machine learning model using the selection and the tags; receiving a second medical record for a second person; scanning the second medical record to generate second tags; and assigning visual indicators to the second tags based on inputting the second tags into the trained machine learning model.

Clause 15: A non-transitory computer readable medium comprising instructions to be executed in a processor, the instructions when executed in the processor perform an operation, the operation comprising: receiving a medical record for a person; scanning the medical record to generate tags, the tags providing information about at least one of medical needs or a responsible payer of the person; assigning visual indicators to the tags to represent a compatibility of the information in the tags with a particular healthcare facility; and transmitting, for display, a GUI comprising (i) the tags along with their visual indicators and (ii) at least a portion of the medical record.

Clause 16: In addition to the computer readable medium of clause 15, wherein assigning the visual indicators to the tags comprises: comparing the information in the tags to facility attributes corresponding to the particular healthcare facility, the facility attributes indicating capabilities or preferences of the particular healthcare facility.

Clause 17: In addition to the computer readable medium of clause 16, wherein the compatibility represented by the visual indicators is defined by whether the particular healthcare facility can satisfy the medical needs of the person based on capabilities or preferences of the particular healthcare facility.

Clause 18: In addition to the computer readable medium of clauses 15, 16, or 17, wherein scanning the medical record to generate tags further comprises: evaluating the medical records using natural language processing or a keyword search to identify text in the medical records that matches, or is a derivative of, a tag stored in a predefined tag database.

Clause 19: In addition to the computer readable medium of clauses 15, 16, 17, or 18, where the operation further comprises: identifying respective portions of the medical record that has text used to generate each of the tags; and converting the tags into selectable tags that, when displayed in the GUI and selected by a user, update the GUI to show the respective portion of the medical record corresponding to a selected one of the tags.

Clause 20: In addition to the computer readable medium of clauses 15, 16, 17, 18, or 19, wherein the operation further comprises: receiving a selection from a user indicating whether the person was recommend to be transferred to the particular healthcare facility; training a machine learning model using the selection and the tags; receiving a second medical record for a second person; scanning the second medical record to generate second tags; and assigning visual indicators to the second tags based on inputting the second tags into the trained machine learning model.

What is claimed is:

1. A method, comprising:
receiving a medical record for a person;
scanning the medical record to generate tags, the tags providing information about at least one of medical needs or a responsible payer of the person;
assigning visual indicators to the tags to represent a compatibility of the information in the tags with a particular healthcare facility; and
transmitting, for display, a GUI comprising (i) the tags along with their visual indicators and (ii) at least a portion of the medical record.

2. The method of claim 1, wherein assigning the visual indicators to the tags comprises:
comparing the information in the tags to facility attributes corresponding to the particular healthcare facility, the facility attributes indicating capabilities or preferences of the particular healthcare facility.

3. The method of claim 2, wherein the compatibility represented by the visual indicators is defined by whether the particular healthcare facility can satisfy the medical needs of the person based on the capabilities or preferences of the particular healthcare facility.

4. The method of claim 1, wherein scanning the medical record to generate tags further comprises:
evaluating the medical records using natural language processing or a keyword search to identify text in the medical records that matches, or is a derivative of, a tag stored in a predefined tag database.

5. The method of claim 1, further comprising:
identifying respective portions of the medical record that has text used to generate each of the tags; and
converting the tags into selectable tags that, when displayed in the GUI and selected by a user, update the GUI to show the respective portion of the medical record corresponding to a selected one of the tags.

6. The method of claim 5, wherein updating the GUI to show the respective portion of the medical record corresponding to the selected one of the tags further comprises at least one of:
auto-scrolling the GUI to a portion that includes the respective portion of the medical record; or
outputting for display a pop-up window containing the respective portion of the medical record.

7. The method of claim 1, further comprising:
receiving a selection from a user indicating whether the person was recommend to be transferred to the particular healthcare facility;
training a machine learning model using the selection and the tags;
receiving a second medical record for a second person;
scanning the second medical record to generate second tags; and
assigning visual indicators to the second tags based on inputting the second tags into the trained machine learning model.

8. A computing system, comprising:
a processor; and
memory storing an application which, when executed by the processor, performs an operation, the operation comprising:
receiving a medical record for a person;
scanning the medical record to generate tags, the tags providing information about at least one of medical needs or a responsible payer of the person;
assigning visual indicators to the tags to represent a compatibility of the information in the tags with a particular healthcare facility; and
transmitting, for display, a GUI comprising (i) the tags along with their visual indicators and (ii) at least a portion of the medical record.

9. The computing system of claim 8, wherein assigning the visual indicators to the tags comprises:
comparing the information in the tags to facility attributes corresponding to the particular healthcare facility, the facility attributes indicating capabilities or preferences of the particular healthcare facility.

10. The computing system of claim 9, wherein the compatibility represented by the visual indicators is defined by whether the particular healthcare facility can satisfy the medical needs of the person based on capabilities or preferences of the particular healthcare facility.

11. The computing system of claim 8, wherein scanning the medical record to generate tags further comprises:
evaluating the medical records using natural language processing or a keyword search to identify text in the medical records that matches, or is a derivative of, a tag stored in a predefined tag database.

12. The computing system of claim 8, where the operation further comprises:
identifying respective portions of the medical record that has text used to generate each of the tags; and
converting the tags into selectable tags that, when displayed in the GUI and selected by a user, update the GUI to show the respective portion of the medical record corresponding to a selected one of the tags.

13. The computing system of claim 12, wherein updating the GUI to show the respective portion of the medical record corresponding to the selected one of the tags further comprises at least one of:
auto-scrolling the GUI to a portion that includes the respective portion of the medical record; or
outputting for display a pop-up window containing the respective portion of the medical record.

14. The computing system of claim 8, wherein the operation further comprises:
receiving a selection from a user indicating whether the person was recommend to be transferred to the particular healthcare facility;
training a machine learning model using the selection and the tags;
receiving a second medical record for a second person;
scanning the second medical record to generate second tags; and
assigning visual indicators to the second tags based on inputting the second tags into the trained machine learning model.

15. A non-transitory computer readable medium comprising instructions to be executed in a processor, the instructions when executed in the processor perform an operation, the operation comprising:
receiving a medical record for a person;
scanning the medical record to generate tags, the tags providing information about at least one of medical needs or a responsible payer of the person;
assigning visual indicators to the tags to represent a compatibility of the information in the tags with a particular healthcare facility; and
transmitting, for display, a GUI comprising (i) the tags along with their visual indicators and (ii) at least a portion of the medical record.

16. The non-transitory computer readable medium of claim 15, wherein assigning the visual indicators to the tags comprises:
comparing the information in the tags to facility attributes corresponding to the particular healthcare facility, the facility attributes indicating capabilities or preferences of the particular healthcare facility.

17. The non-transitory computer readable medium of claim 16, wherein the compatibility represented by the visual indicators is defined by whether the particular healthcare facility can satisfy the medical needs of the person based on capabilities or preferences of the particular healthcare facility.

18. The non-transitory computer readable medium of claim 15, wherein scanning the medical record to generate tags further comprises:
evaluating the medical records using natural language processing or a keyword search to identify text in the medical records that matches, or is a derivative of, a tag stored in a predefined tag database.

19. The non-transitory computer readable medium of claim 15, where the operation further comprises:
identifying respective portions of the medical record that has text used to generate each of the tags; and
converting the tags into selectable tags that, when displayed in the GUI and selected by a user, update the GUI to show the respective portion of the medical record corresponding to a selected one of the tags.

20. The non-transitory computer readable medium of claim 15, wherein the operation further comprises:
receiving a selection from a user indicating whether the person was recommend to be transferred to the particular healthcare facility;
training a machine learning model using the selection and the tags;
receiving a second medical record for a second person;
scanning the second medical record to generate second tags; and
assigning visual indicators to the second tags based on inputting the second tags into the trained machine learning model.
